(19) United States
(12) Patent Application Publication
(10) Pub. No.: US 2021/0128283 A1
(43) Pub. Date: May 6, 2021

(54) DENTURE BLOCK

(71) Applicant: GC CORPORATION, Bunkyo-ku (JP)

(72) Inventors: Hiroki KATO, Itabashi-ku (JP); Takayuki MICHII, Itabashi-ku (JP)

(73) Assignee: GC CORPORATION, Bunkyo-ku (JP)

(21) Appl. No.: 16/639,464

(22) PCT Filed: Aug. 15, 2018
(86) PCT No.: PCT/JP2018/030374

§ 371 Date: Feb. 14, 2020

(30) Foreign Application Priority Data
Aug. 16, 2017 (JP) 2017-157282

(51) Int. Cl. A61C 13/00

(52) U.S. Cl. CPC A61C 13/0004; A61C 13/0022; A61C 13/0007

(57) ABSTRACT

The present invention provides a denture block capable of integrally and simply manufacturing a denture by using a CAD/CAM system. A denture is manufactured by cutting using a CAD/CAM system in a state in which the ridge portion 1b is gripped, has: an upper part which is a white part 2 to correspond to a tooth part of the denture; and a lower part which is a gingival color part 3 to correspond to a denture plate of the denture, wherein the white part 2 and the gingival color part 3 are integrally formed by being brought into close contact with each other with no gaps therebetween including the inside thereof, and a plurality of convex portions 4 and concave portions 5 are formed on an upper surface 3a which is the boundary surface of the gingival color part 3 with the white part 2.

### TECHNICAL FIELD

The present invention relates to a denture block used for manufacturing dentures in a dental field.

### BACKGROUND ART

In a dental field, there has been proposed a method for manufacturing an orthoprosthesis or a (plate) denture by cutting or polishing a block-shaped material using a CAD/CAM system (for example, see Patent Literatures 1 to 3).

Among these, there are methods for easily manufacturing an orthoprosthesis by imparting a gradient to hardness or strength inside a block-shaped material (for example, see Patent Literatures 1 and 2), but these Patent Literatures do not mention the denture base, and do not mention at all about the manufacture of the denture from these materials.

Further, a method for manufacturing a denture by cutting and polishing a resin material using a CAD/CAM system has been proposed (for example, see Patent Literature 3).

As the method for manufacturing a denture, there have been proposed a method for separately manufacturing a denture base and an artificial tooth using a CAD/CAM system and then bonding an artificial tooth to a denture base, and a method for integrally forming a denture base and an artificial tooth (for example, see Patent Literature 3).

When the denture base and the artificial tooth are manufactured separately, the artificial tooth needs to be arranged and bonded to the denture base accurately, which significantly lowers the manufacturing efficiency. On the other hand, when the denture base and the artificial tooth are integrally molded, the bonding step is unnecessary, but a step of individually coloring the denture base and the artificial tooth is required, and even in this case, it is difficult to say that the manufacturing efficiency is high (for example, see Patent Literature 3, paragraph 0050)

### CITATION LIST

### Patent Literature

Patent Literature 1: WO 2014/034736 A1
Patent Literature 2: JP 2012-187357 A
Patent Literature 3: WO 2010/058822 A1

### SUMMARY OF INVENTION

### Technical Problem

The present invention has been made in view of the above problems, and relates to a denture block capable of integrally and simply manufacturing the denture (denture base and an artificial tooth) using a CAD/CAM system.

### Solution to Problem

As a result of intensive research to solve the above problems, and as a result, the present inventors have found that if a denture block is adopted in which a ridge portion is formed at a vertical central portion of a side surface of a cylinder, an upper part is a pre-colored white part serving as a tooth part of a denture, and a lower part is a pre-colored gingival color part serving as a denture base part, the denture can be integrally manufactured by being cut using a CAD/CAM system in a state in which the ridge portion is gripped, since the upper part and the lower part are divided into two colors in advance, a coloring step can be significantly reduced during the manufacturing of the denture and manufacturing efficiency becomes extremely high, if the white part and the gingival color part are integrally formed by being brought into close contact with each other with no gap therebetween including an inside thereof, the higher strength and durability can be imparted compared to the case where the artificial tooth is bonded and fixed to the denture base, and furthermore, if a plurality of convex portions and concave portions are formed on an upper surface that is a boundary surface between the gingival color part and the white part, it is easy to express a boundary part between the tooth part and the denture base part of the denture and it is possible to reduce a fine color tone correction step of the boundary part, and completed the present invention.

That is, according to the present invention, in the denture block in which the ridge portion is formed in the vertical central portion of the side surface of the cylinder, the upper part is the white part serving as the tooth part of the denture, the lower part is the gingival color part serving as the denture base part of the denture, and the denture is manufactured by being cut by the CAD/CAM system in the state in which the ridge portion is gripped, the white part and the gingival color part are integrally formed by being brought into close contact with each other with no gap therebetween including the inside thereof, and the plurality of convex portions and concave portions are formed on the upper surface that is the boundary surface between the gingival color part and the white part.

If the convex portion of the upper surface of the gingival color part is a convex portion protruding in a prismatic shape or the concave portion of the upper surface of the gingival color part is a concave portion recessed in the prismatic shape, the denture block according to the present invention can be easily manufactured, and furthermore, if the upper surface of the gingival color part has a wavy curved surface by the convex portion and the concave portion, it is preferable because it is easy to express the complicated curved state of the boundary part between the tooth part of the denture and the gingival part and it is possible to further reduce the number of color tone correction steps.

Furthermore, if the convex portions of the upper surface inside the gingival color part have the same shape or the concave portions of the upper surface inside the gingival color part have the same shape, and the convex portions of the upper surface inside the gingival color part are arranged at equal intervals or the concave portions of the upper surface inside the gingival color part are arranged at equal intervals, it was investigated that the versatility of the denture block is high, and the denture block is convenient to use and preferred.

### Advantageous Effects of Invention

In the denture block according to the present invention, since the ridge portion is formed at the vertical central portion of the side surface of the cylinder, the upper part is the pre-colored white part serving as the tooth part of the denture, and the lower part is the pre-colored gingival color part serving as the denture base part, the denture can be integrally manufactured by being cut using the CAD/CAM system in the state in which the ridge portion is gripped, since the upper part and the lower part are divided into two colors in advance, the coloring step can be significantly reduced during the manufacturing of the denture and the manufacturing efficiency becomes extremely high, since the white part and the gingival color part are integrally formed by being brought into close contact with each other with no gap therebetween including an inside thereof, the higher strength and durability can be imparted compared to the case where the artificial tooth is bonded and fixed to the denture base, and furthermore, if the plurality of convex portions and concave portions are formed on the upper surface that is the boundary surface between the gingival color part and the white part, it is easy to express the boundary part between the tooth part and the denture base part of the denture, and it is possible to reduce the fine color tone correction step of the boundary part.

In the state in which the convex portion of the upper surface of the gingival color part is the convex portion protruding in the prismatic shape or the concave portion of the upper surface of the gingival color part is the concave portion recessed in the prismatic shape, it is preferable that the denture block according to the present invention can be easily manufactured, and furthermore, in the state in which the upper surface of the gingival color part is the wavy curved surface by the convex portion and the concave portion, it is preferable because it is easy to express the complicated curved state of the boundary part between the tooth part of the denture and the gingival part and it is possible to further reduce the color tone correction step.

Furthermore, in the state in which the convex portions of the upper surface inside the gingival color part have the same shape or the concave portions of the upper surface inside the gingival color part have the same shape, and the convex portions of the upper surface inside the gingival color part are arranged at equal intervals or the concave portions of the upper surface inside the gingival color part are arranged at equal intervals, the versatility of the denture block is high, and the denture block is convenient to use and preferred.

### BRIEF DESCRIPTION OF DRAWINGS

FIG. 1 is a front view showing an aspect of a denture block according to the present invention.

FIG. 2 is a plan view of the denture block in FIG. 1.

FIG. 3 is a bottom view of the denture block in FIG. 1.

FIG. 4 is a cross-sectional view taken along line A-A in FIG. 1.

FIG. 5 is a cross-sectional view taken along line B-B of FIG. 2.

FIG. 6 is a cross-sectional view taken along line C-C of FIG. 2.

FIG. 7 is a front view showing another aspect of a denture block according to the present invention.

FIG. 8 is a plan view of the denture block in FIG. 7.

FIG. 9 is a cross-sectional view taken along line D-D in FIG. 7.

FIG. 10 is a cross-sectional view taken along line E-E in FIG. 8.

FIG. 11 is a cross-sectional view taken along line F-F in FIG. 8.

FIG. 12 is a front view showing still another aspect of a denture block according to the present invention.

FIG. 13 is a plan view of the denture block in FIG. 12.

FIG. 14 is a cross-sectional view taken along line G-G of FIG. 12.

FIG. 15 is a cross-sectional view taken along line H-H in FIG. 13.

FIG. 16 is a cross-sectional view taken along line I-I in FIG. 13.

### DESCRIPTION OF EMBODIMENTS

Hereinafter, a denture block according to the present invention will be described in detail.

The denture block according to the present invention has a two-layer structure of a “white part 2 serving as a tooth part of the denture” and a “gingival color part 3 serving as a denture base part of the denture”. The white part 2 and the gingival color part 3 may be made of the same material or different materials. Even if the white part 2 and the gingival color part 3 are made of different materials, a sufficient adhesive force can be expected because a fitting portion has irregularities.

<Resin-Based Material>

As a resin-based material used for the “white part 2 serving as the tooth part of the denture” or the “gingival color part 3 serving as the denture base part of the denture”, an acrylic resin obtained by polymerizing PMMA or MMA can be preferably used.

In addition, a composite resin can also be used favorably. As a (meth)acrylate compound used for the composite resin, methacrylate or acrylate monomers, or oligomers or prepolymers can be used, and are known as substances used for dental materials. More specifically, examples of the (meth)acrylate compound include methyl(meth)acrylate, ethyl(meth)acrylate, isopropyl(meth)acrylate, n-butyl(meth)acrylate, isobutyl(meth)acrylate, hydroxypropyl(meth)acrylate, tetrahydrofurfuryl(meth)acrylate, glycidyl(meth)acrylate, 2-hydroxyethyl(meth)acrylate, 2-hydroxypropyl(meth)acrylate, 3-hydroxypropyl(meth)acrylate, 2-ethoxyethyl(meth)acrylate, 2-methoxyethyl(meth)acrylate, 2-ethylhexyl(meth)acrylate, benzyl(meth)acrylate, 2-hydroxy-1,3-di(meth)acryloxypropane, ethylene glycol di(meth)acrylate, diethylene glycol di(meth)acrylate, triethylene glycol di(meth)acrylate, butylene glycol di(meth)acrylate, neopentyl glycol di(meth)acrylate, 1,3-butanediol di(meth)acrylate, 1,4-butanediol di(meth)acrylate, 1,6-hexanediol di(meth)acrylate, trimethylolpropane tri(meth)acrylate, trimethylol methane tri(meth)acrylate, pentaerythritol tri(meth)acrylate, and trimethylolmethane tri(meth)acrylate, pentaerythritol tetra(meth)acrylate, polybutylene glycol di(meth)acrylate, bisphenol A polyethoxy methacrylate, bisphenol A diglycidyl(meth)acrylate, and the like.

Further, examples of (meth)acrylate having a urethane bond includes di-2-(meth)acryloxyethyl-2,2,4-trimethylhexamethylenedicarbamate, 1,3,5-tris[1,3-bis{(meth)acryloyloxy}-2-propoxycarbonylaminohexane]-1,3,5-(1H, 3H, 5H)triazine-2,4,6-trione, urethane oligomer(meth)acrylate made of other 2,2′-di(4-hydroxy cyclohexyl)propane, urethane oligomer(meth)acrylate made of 2-oxypanone, hexamethylene diisocyanate, and 2-hydroxyethyl(meth)acrylate, 1,3-butanediol, hexamethylene diisocyanate, and 2-hydroxyethyl(meth)acrylate, and the like. Examples of a filler component include silica glass, barium glass, strontium glass, and the like.

<Zirconia and Ceramic-Based Materials>

As a material used for the “white part 2 serving as the tooth part of the denture” or the “gingival color part 3 serving as the denture base part of the denture”, zirconia (zirconium oxide) can be preferably used.

In addition, ceramic can be used, and in particular, porcelain-based and lithium disilicate-based ceramic can be preferably used.

For each of the above materials, materials colored in advance according to a part (white part 2 or gingival color part 3) to be used are used. A coloring agent is not particularly limited as long as the coloring agent is harmless to the human body.

In addition, the above-described materials are an example, and the material of the denture block according to the present invention is not limited to these materials.

Hereinafter, a step of manufacturing a denture block according to the present invention will be described in detail.

“Preparation of Materials Constituting Denture Block”

An acrylic resin having a blending ratio of 50% by mass of PMMA and 50% by mass of MMA was used as the “resin” used for manufacturing the denture block according to the present invention.

Further, as a “composite resin”, a resin obtained by adding a trace amount of polymerization initiator to 20% by mass of UDMA, 20% by mass of NPG, and 60% by mass of barium glass filler was used.

As the “ceramic”, “lithium disilicate ceramic” or “porcelain-based ceramic” was used. As the “lithium disilicate ceramic”, a simple substance (SiO₂: 63.0% by mass, Li₂O: 19.6% by mass, Al₂O₃: 5.1% by mass, K₂O: 2.7% by mass, MgO: 2.1% by mass, BaO: 1.3% by mass, P₂O₅: 4.1% by mass, ZrO₂: 2.1% by mass) of lithium silicate was used. In addition, as the “porcelain-based ceramic”, a simple substance (20% by mass of potassium oxide, 30% by mass of aluminum oxide, and 50% by mass of silicon dioxide) of leucite-based crystallized glass powder was used. Further, as the “zirconia”, a simple substance of zirconium oxide was used.

### EXAMPLES

“Manufacture of Denture Block According to the Present Invention”

Since a denture block according to the present invention has a two-layer structure of “white part 2 serving as a tooth part of a denture” and “gingival color part 3 serving as a denture base part of the denture”, one of the white part 2 and the gingival color part 3 is molded first and the other thereof is subsequently molded to complete the whole denture block. At this time, either the “white part 2” or the “gingival color part 3” may also be molded first.

### Example 1

### Combination of Acrylic Resin and Acrylic Resin

The acrylic resin prepared and colored as described above was filled in a mold, and one of the “white part 2” or the “gingival color part 3” was molded by heat polymerization or photopolymerization. Thereafter, the denture block according to the present invention was manufactured by pouring an acrylic resin having a different color into an upper surface of one of the “white part 2” or the “gingival color part 3” first molded, and heat-polymerizing or photopolymerizing the other of the “white part 2” or the “gingival color part 3”.

At this time, in the denture block according to the present invention, a plurality of convex portions and concave portions are formed on a boundary surface between the “white part 2” and the “gingival color part 3”, and a contact area is large at the “white part 2” and the “gingival color part 3”, so it is also possible to integrate the “white part 2” and the “gingival color part 3” simply by polymerizing the poured acrylic resin later.

However, since it may not necessarily obtain a sufficient adhesive force depending on the number and shapes of convex portions and concave portions, if the upper surface of one of the “white part 2” and the “gingival color part 3” first molded is subjected to roughening processing by sandblasting or the like, applied with an adhesive, such as a denture primer or a resin primer, as an adhesive, and then the acrylic resin is poured into the other of the “white part 2” and the “gingival color part 3”, it is preferable because a sufficient adhesive force can be obtained.

### Example 2

### Combination of Composite Resin and Composite Resin

The composite resin prepared and colored as described above was filled in a mold, and one of the “white part 2” or the “gingival color part 3” was molded by heat polymerization or photopolymerization. Thereafter, the denture block according to the present invention was manufactured by performing, by sandblasting or the like, roughening processing on an upper surface of one of the “white part 2” or the “gingival color part 3” first molded, applying a ceramic primer as an adhesive to the upper surface, and pouring a composite resin having a different color into the upper surface, and molding the other of the “white part 2” or the “gingival color part 3” by the heat polymerization or the photopolymerization.

### Example 3

### Combination of Ceramic and Ceramic

The ceramic resin prepared and colored as described above was filled in a mold, and one of the “white part 2” or the “gingival color part 3” was molded. Thereafter, the denture block according to the present invention was manufactured by pouring “lithium disilicate-based ceramic” having a different color into an upper surface of one of the “white part 2” or the “gingival color part 3” first molded, kneading “porcelain-based ceramic”, and molding the other side of the “white part 2” or the “gingival color part 3.”

Note that in the combination of ceramic and ceramic, for example, when the “lithium disilicate-based ceramic” is poured later, a melting temperature thereof is high, and even when the “ porcelain-based ceramic” is kneaded and fired, the temperature becomes high, and therefore, the upper surface of one of the “white part 2” or the “gingival color part 3” first molded does not required to be applied with, in particular, an adhesive because a surface layer thereof is melted.

### Example 4

### Combination of Zirconia and Zirconia

The zirconia powder prepared and colored as described above was filled in a mold and temporarily sintered, and one of a “white part 2” or a “gingival color part 3” was molded. Thereafter, the denture block according to the present invention was manufactured by filling a zirconia powder having a different color in an upper surface of one of the “white part 2” or the “gingival color part 3” first molded and temporarily sintering the zirconia, and molding the other of the “white part 2” or the “gingival color part”.

### Example 5

### Combination of Composite Resin and Acrylic Resin

The composite resin prepared and colored as described above was filled in a mold, and one of a “white part 2” or a “gingival color part 3” was molded. Thereafter, the denture block according to the present invention was manufactured by performing, by sandblasting or the like, roughening processing on an upper surface of one of the “white part 2” or the “gingival color part 3” first molded, applying a ceramic primer as an adhesive to the upper surface, and pouring an acrylic resin having a different color into the upper surface, and molding the other of the “white part 2” or the “gingival color part 3” by heat polymerization or photopolymerization.

### Example 6

### Combination of Ceramic and Acrylic Resin or Composite Resin

The acrylic resin prepared and colored as described above was filled in a mold, and one of a “white part 2” or a “gingival color part 3” was molded. Thereafter, the denture block according to the present invention was manufactured by performing, by sandblasting or the like, roughening processing on an upper surface of one of the “white part 2” or the “gingival color part 3” first molded, applying a ceramic primer as an adhesive to the upper surface, and pouring an acrylic resin or a composite resin having a different color into the upper surface, and molding the other of the “white part 2” or the “gingival color part 3” by heat polymerization or photopolymerization.

“Convex Portion and Concave Portion of Denture Block According to the Present Invention”

In the denture block according to the present invention, since a plurality of convex portions 4 and concave portions 5 are formed on an upper surface 3a of the gingival color part 3, it is easy to express the boundary part between the tooth part and the denture base part of the denture, and it is possible to greatly reduce labor or the like for finishing the boundary part.

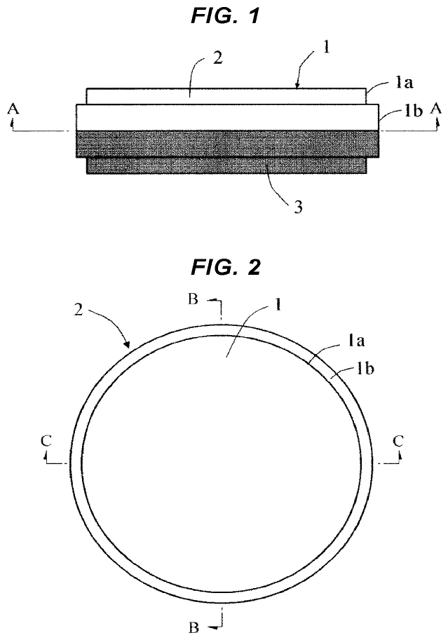

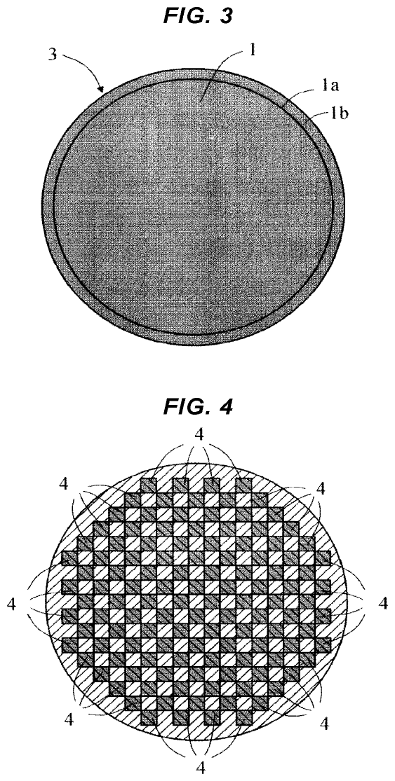

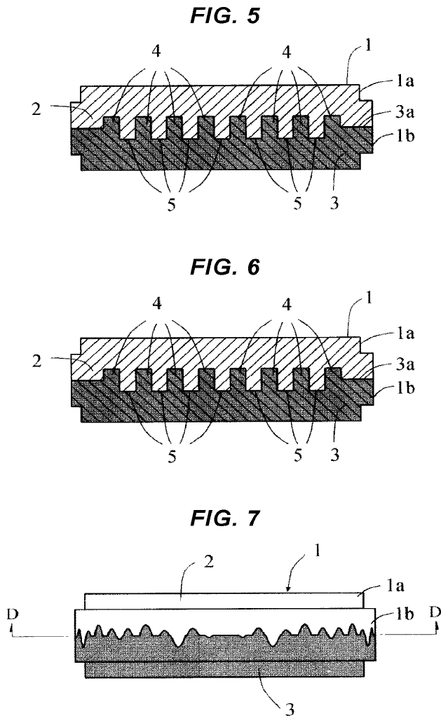

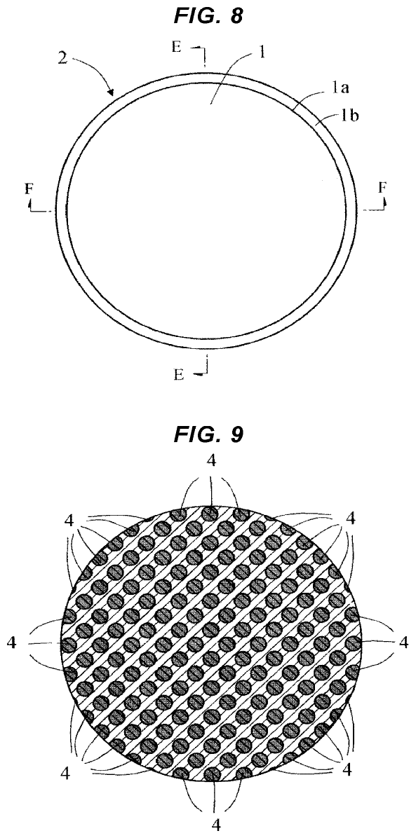

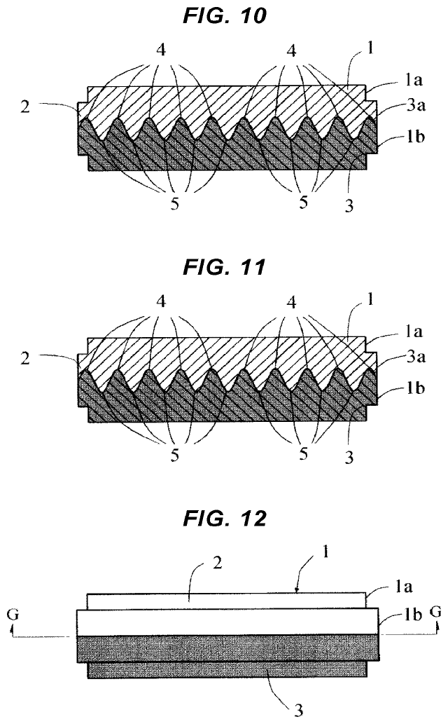

In the denture block according to the present invention, as in the above-described examples, since one of the “white part 2” or the “gingival color part 3” may be molded first, for example, when the “gingival color part 3” may be molded first. In the case of molding, a mold is prepared so that the convex portion 4 and the concave portion 5 having a prismatic shape as shown in FIGS. 1 to 6 are formed on the upper surface 3a of the gingival color part 3, and even when the convex portion 4 and the concave portion 5 are a wavy curved surface as shown in FIGS. 7 to 11, a mold corresponding thereto is prepared, so the “gingival color part 3” may be molded by pouring and filling the above-described materials into these molds.

Thereafter, if the above-described materials constituting the “white part 2” is poured and filled into the upper surface 3a of the “gingival color part 3” to form the “white part 2”, the concave portion or the convex portion that fit with the convex portion 4 or the concave portion 5 of the “gingival color part 3” can also be easily formed on the “white part 2”.

As in the case where the “white part 2” is molded first, the mold is used so that the concave portion or the convex portion is formed on the upper surface of the “white part 2” to first mold the “white part 2”, and if the “gingival color part 3” is molded by pouring and filling the above-described materials constituting the “gingival color part 3” into the upper surface of the “ white part 2”, the convex portion 4 or the concave portion 5 of the “gingival color part 3” can be easily formed.

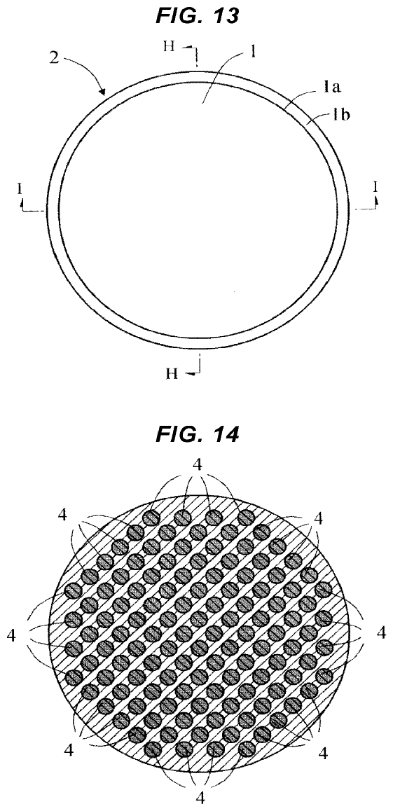

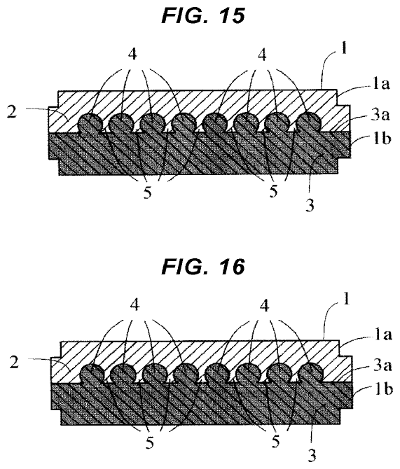

Next, in the aspects shown in FIGS. 12 to 16, the convex portion 4 is almost spherical, so the upper surface 3a side of the gingival color part 3 is thin, and is in an undercut state. When the “gingival color part 3” having such a shape is molded with a mold, it is necessary to break the mold when the “gingival color part 3” is removed from the mold, which makes mass production difficult and causes a big manufacturing problem.

In such a case, the gingival color part 3 having a flat upper surface 3a is first manufactured using a mold or the like, and a substantially spherical convex portion 4 is separately manufactured and then the substantially spherical convex portion 4 is fixed to the flat upper surface 3a of the gingival color part 3 to mold the whole gingival color part 3. Thereafter, as in the above examples, if the material of white part 2 is poured and filled and molded, the denture block according to the present invention having the undercut portion as shown in FIGS. 12 to 16 can be easily manufactured and mass-produced.

“Arrangement of Convex Portion and Concave Portion of Denture Block According to the Present Invention”

The convex portion 4 or the concave portion 5 of the gingival color part 3 or the concave portion or the convex portion of the white part 2 formed so as to be fitted with the convex portion 4 and the concave portion 5 are similar to the aspects of FIGS. 1 to 6 or the aspects of FIGS. 12 to 16 and may be formed only in the denture block according to the present invention, but as in the aspects of FIGS. 7 to 11, the convex portion or the concave portion may also be formed over the whole of the boundary part between the white part 2 and the gingival color part 3 so that the convex portion 4 or the concave portion 5 of the gingival color part 3 or the concave portion or the convex portion of the white part 2 appears on a side surface 1a of the denture block according to the present invention, and at that time, the convex portion or the concave portion does not necessarily remain in a complete shape on the side surface 1a and may be formed in a state in which a part of the convex portion 4 or the concave portion 5 is missing according to the shape of the side surface 1a.

In this way, if the plurality of convex portions and concave portions are formed up to the side surface 1a so that the convex portion or the concave portion is on the side surface 1 a, when the denture is manufactured by the denture block according to the present invention, it is more preferable because the denture can be used up to near the side surface 1a without waste.

Note that in each of the cross-sectional views of FIGS. 4, 9, and 14, the number of convex portions 4 is large, and when all the convex portions 4 are drawn with lead lines and attached with reference numerals, the drawing may become unclear, and therefore, in each of the above cross-sectional views, the lead lines or reference numerals are omitted for the convex portion 4 located at the center.

“Manufacture of Denture by Denture Block According to the Present Invention”

The denture block according to the present invention is for manufacturing a total denture or a partial denture using the CAD/CAM system. Therefore, after a state in an oral cavity of a patient is obtained as three-dimensional data, the denture block according to the present invention is cut and polished based on the three-dimensional data to manufacture the denture.

“Finishing Step of Manufacture of Denture by Denture Block According to the Present Invention”

If the CAD/CAM system is used as described above, the intended shape can be manufactured, but a color tone needs to be adjusted. For example, a portion corresponding to a cervical area or a crown papilla may not have a desired color tone. Therefore, if the cervical area is not a crown color and the crown papilla is not a gingival color, the correction is made using a resin, a composite resin, and porcelain. In addition, even in the case where the denture block according to the present invention has the concave portion and the convex portion, which may appear at unintended positions and may not obtain the desired color tone, the same correction is performed. In this way, the correction is performed, and then the polishing and finishing are performed as needed.

Note that when it is necessary to match the color tone with other natural teeth in the partial denture or the like, coloring may be performed using a stain material.

Specific finishing steps in each example are as follows. First, in Example 1 (combination of acrylic resin and acrylic resin), when an acrylic resin part is corrected with the resin-based material, a denture primer or a resin primer may be applied as an adhesive. Then, the acrylic resin part is corrected using the resin-based material of the crown color or the gingival color, and is polymerized and cured. In addition, when the acrylic resin part is corrected with the composite resin-based material, a surface thereof is roughened by the sandblasting or the like, applied with a Zenopal primer as an adhesive, and irradiated with light. Thereafter, the acrylic resin part is corrected using the composite resin-based material of the crown color or the gingival color, and polymerized and cured, and the surface thereof is polished as needed.

In Example 2 (combination of composite resin and composite resin), when a composite resin part is corrected with the resin-based material or the composite resin-based material, after a surface of the composite resin part is roughened by the sandblasting or the like and applied with a ceramic primer as an adhesive, the composite resin part is corrected using the resin-based material or the composite resin-based material of the crown color or the gingival color and polymerized and cured, and a surface thereof is polished as needed.

In Example 3 (combination of ceramic and ceramic), when a ceramic part is corrected with the resin-based material or the composite resin-based material, a surface thereof is roughened by the sandblasting or the like, applied with the ceramic primer as an adhesive, and then corrected using the resin-based material or the composite resin-based material of the crown color or the gingival color and polymerized and cured, and a surface thereof is polished as needed.

In Example 4 (combination of zirconia and zirconia), when a zirconia part is corrected with the resin-based material or the composite resin-based material, a surface thereof is roughened by the sandblasting or the like, applied with a metal primer Z as an adhesive, and then corrected using the resin-based material or the composite resin-based material of the crown color or the gingival color and polymerized and cured, and a surface thereof is polished as needed.

In Example 5 (combination of composite resin and acrylic resin), when a composite resin part is corrected with the resin-based material or the composite resin-based material, a surface thereof is roughened by the sandblasting or the like, applied with the ceramic primer as an adhesive, and then corrected using the resin-based material or the composite resin-based material of the crown color or the gingival color and polymerized and cured. In addition, when the acrylic resin part is corrected with the resin-based material, the denture primer or the resin primer may be applied as an adhesive. Then, the acrylic resin part is corrected using the resin-based material of the crown color or the gingival color, and is polymerized and cured. In addition, when the acrylic resin part is corrected with the composite resin-based material, a surface thereof is roughened by the sandblasting or the like, applied with the Zenopal primer as an adhesive, and irradiated with light. Thereafter, the acrylic resin part is corrected using the composite resin-based material of the crown color or the gingival color, and polymerized and cured, and the surface thereof is polished as needed.

In Example 6 (combination of ceramic and acrylic resin or composite resin), when a ceramic part is corrected with the resin-based material or the composite resin-based material, a surface thereof is roughened by the sandblasting or the like, applied with the ceramic primer as an adhesive, and then corrected using the resin-based material or the composite resin-based material of the crown color or the gingival color and polymerized and cured. In addition, when the composite resin part is corrected with the resin-based material or the composite resin-based material, a surface thereof is roughened by the sandblasting or the like, applied with the ceramic primer as an adhesive, and then corrected using the resin-based material or the composite resin-based material of the crown color or the gingival color and polymerized and cured. In addition, when the acrylic resin part is corrected with the resin-based material, the denture primer or the resin primer may be applied as an adhesive. Then, the acrylic resin part is corrected using the resin-based material of the crown color or the gingival color, and is polymerized and cured. In addition, when the acrylic resin part is corrected with the composite resin-based material, a surface thereof is roughened by the sandblasting or the like, applied with the Zenopal primer as an adhesive, and irradiated with light. Thereafter, the acrylic resin part is corrected using the composite resin-based material of the crown color or the gingival color, and polymerized and cured, and the surface thereof is polished as needed.

In addition, when Example 3 (combination of ceramic and ceramic) or Example 4 (combination of zirconia and zirconia) is corrected with a porcelain-based material, the porcelain-based material of the crown color or the gingival color is kneaded and fired and a surface thereof is polished as needed. Note that in the case of Example 6 (a combination of ceramic and acrylic resin or composite resin), if the Example 6 is corrected with the porcelain-based material and fired, the acrylic resin or the composite resin would burn, so the Example 6 cannot be corrected with the porcelain-based material.

As described above, the denture block according to the present invention can be easily made of various materials depending on the application and the like, and also has the two-layer structure having different color tones, and has the convex portion and the concave portion provided on the boundary surface, so it is possible to greatly reduce the time and effort required for adjusting the color tone and the like at the time of manufacturing the denture, and it is possible to easily manufacture the denture having excellent aesthetics.

### REFERENCE SIGNS LIST

1 Cylinder

1a Side surface

1b Ridge portion

2 White part

3 Gingival color part

3a Upper surface

4 Convex portion

5 Concave portion

1. A denture block, comprising:
a cylinder having a side surface and including a ridge portion formed in a vertical central portion of the side surface, the cylinder having a white part in an upper portion thereof and a gingival color part in a lower portion thereof,
wherein the white part is to be a tooth part of a denture,
the gingival color part is to be a denture base part of the denture,
the ridge portion is to be a grip for holding the denture block in manufacturing the denture by a CAD/CAM system,
the white part and the gingival color part are integrally formed and have no gap therebetween including an inside thereof, and
a plurality of convex portions and concave portions are formed on an upper surface of the gingival color part, which forms a boundary between the gingival color part and the white part.

2. The denture block according to claim 1, wherein the convex portions include a convex portion protruding in a prismatic shape.

3. The denture block according to claim 1, wherein the concave portions include a concave portion recessed in the prismatic shape.

4. The denture block according to claim 1, wherein the upper surface of the gingival color part is a wavy curved surface formed by the convex portions and the concave portions.

5. The denture block according to claim 1, wherein the convex portions have the same shape.

6. The denture block according to claim 1, wherein the concave portions have the same shape.

7. The denture block according to claim 1, wherein the convex portions are positioned at equal intervals.

8. The denture block according to claim 1, wherein the concave portions are positioned at equal intervals.
